(19) United States
(12) Patent Application Publication
(10) Pub. No.: US 2008/0112849 A1
(43) Pub. Date: May 15, 2008

(54) MICRO TOTAL ANALYSIS CHIP AND MICRO TOTAL ANALYSIS SYSTEM

(75) Inventors: Kusunoki Higashino, Osaka (JP); Akihisa Nakajima, Tokyo (JP); Yasuhiro Sando, Amagasaki-shi (JP); Youichi Aoki, Tokyo (JP)

Correspondence Address: CANTOR COLBURN, LLP, 20 Church Street, 22nd Floor, Hartford, CT, 06103, US

(73) Assignee: KONICA MINOLTA MEDICAL & GRAPHIC, INC., Tokyo (JP)

(21) Appl. No.: 11/935,529

(22) Filed: Nov. 6, 2007

(30) Foreign Application Priority Data
Nov. 10, 2006 (JP) 2006-304953

(51) Int. Cl. G01N 33/00

(52) U.S. Cl. 422/68.1

(57) ABSTRACT

A micro total analysis chip including: a main flow chip for feeding a liquid; and a divided flow path for dividing and feeding the liquid at a predetermined division ratio, each divided path of the plurality of divided paths having a high flow path narrower than a preceding part and a subsequent part of the each divided path, wherein a flow portion of a first divided path in the divided paths satisfies and expression of: R×Q>σ×L/S, where, Q is a flow rate of the first divided path, S is a sectional area and L is a sectional circumferential length of a flow path of other divided path of the plurality of divided paths, and σ is a surface tension liquid.

### CROSS-REFERENCE TO RELATED APPLICATIONS

The present application is based on Japanese Patent Application No. 2006-304953 filed with Japanese Patent Office on Nov. 10, 2006, the entire content of which is hereby incorporated by reference.

### BACKGROUND OF THE INVENTION

1. Field of the Invention

The present invention relates to a micro total analysis chip and a micro total analysis system and more particularly to a micro total analysis chip and a micro total analysis system having divided flow paths branched into a plurality of parts for dividing and feeding a liquid such as a specimen or a reagent at a predetermined division ratio.

2. Description of the Related Art

In recent years, by freely using the micro machine art and microfine processing technique for making an apparatus and a means (for example, a pump, a valve, a flow path, a sensor, etc.) for executing the conventional sample adjustment, chemical analysis, and chemical synthesis fine and integrating them on one micro total analysis chip has been developed.

The micro total analysis chip may be called a μ-TAS (micro total analysis system), a bioreactor, a lab-on-chips, or a biochip and is expected to be widely used in the medical examination and diagnosis field, environment measurement field, and agricultural product manufacture field. Actually, as seen in the gene examination, when a complicated step, a skilled act, and an operation of an apparatus are required, the micro chemical analysis system which is automated, speeded up, and simplified enables analysis not only being preferable in cost, necessary sample amount, and necessary time but also executable at any time and place, thus it may be said that its beneficence is great.

In the analysis and examination using such a micro total analysis chip, it is important to shorten the time required for analysis by dividing a specimen into a plurality of parts and making each part react with a different reagent, that is, producing a plurality of reactions in parallel. Furthermore, in the quantitative analysis and examination, it is important to mix a specimen and a reagent at an accurate mixing ratio to react with each other and for that purpose, a method for dividing the specimen and reagent at an accurate division ratio is important.

In the conventional micro total analysis chip, as an art for dividing a liquid, the separation method for a solution after reaction by the so-called two-phase distribution method is known and for example, in Unexamined Japanese Patent Application Publication No. 2001-281233 (JPA2001-281233), the method for distributing fine articles dissolved in the solution by using the difference in the solubility between the two-phase stream layers flowing in parallel, thereby permitting the two-phase streams to react in the separation state without being mixed, and separating the layers from each other at the branch portion to flow them to the branch portion is proposed.

Or, in Unexamined Japanese Patent Application Publication No. 2005-331286 (JPA2005-331286), the method for feeding a liquid to the branch portion in the state that the interface between the two-phase streams is kept stable by treating the inner surface of the flow path, and branching stably the liquids at the branch portion is proposed.

However, the methods described in both of JPA2001-281233 and JPA2005-331286 are methods for separating the liquids between the layers after reaction by the two-phase distribution method and as mentioned above, the separation of the specimen and reagent at the accurate division ratio is not supposed. Particularly, when the division ratio is not one to one, even if the flow path is divided simply into two parts, a required division ratio cannot be obtained, and a new method is required.

Furthermore, in the micro total analysis chip, the sectional dimensions of the flow path are very fine, so that the effect of the interaction such as the capillary force between the inner wall surface of the flow path and the fluid is great. The force is extremely apt to be influenced by the surface state (surface roughness and foreign substances on the surface) of the inner wall surface of the flow path and for example, even if the inner surface is treated as indicated in JPA2005-331286, under the influence thereof, the division ratio is easily varied and to realize an accurate division ratio including a division ratio of one to one, any new countermeasure is necessary.

The present invention was developed with the foregoing in view and is intended to provide a micro total analysis chip and a micro total analysis system having divided flow paths branched into a plurality of parts capable of dividing accurately and feeding a liquid such as a specimen or a reagent at a predetermined division ratio for producing a plurality of reactions in parallel, thereby shortening the time required for analysis.

### SUMMARY

The object of the present invention can be accomplished by use of the constitution indicated below.

(1) A micro total analysis chip including: a main flow path for feeding a liquid; and a divided flow path branched into a plurality of divided paths for dividing and feeding the liquid fed from the main flow path at a predetermined division ratio, each divided path of the plurality of divided paths having a high flow path resistance portion comprising a narrowed down flow path narrower than a preceding part and a subsequent part of the each divided path, wherein a flow path resistance R of the high flow path resistance portion of a first divided path in any of the plurality of divided paths satisfies an expression of:

R×Q>σ×L/S

where, Q is a flow rate of the first divided path, S is a sectional area and L is a sectional circumferential length of a flow path of other divided path of the plurality of divided paths than the first divided path, and a is a surface tension of the liquid.
(2) A micro total analysis chip stated in Item (1), characterized in that each of the plurality of divided paths has at least one water repellent valve, wherein the flow path resistance R of the high flow path resistance portion of the first divided path in any of the plurality of divided paths satisfies an expression of:

R×Q>P

where, Q is the flow rate of the first divided path, P is an upper limit of a liquid holding pressure of the water repellent valve in other divided path of the plurality of divided paths than the first divided path.
(3) A micro total analysis system including: a micro total analysis chip stated in (1) or (2); a liquid feed apparatus connected to the micro total analysis chip for feeding a liquid in the micro total analysis chip; and a detector for detecting a target material generated on the micro total analysis chip.

### BRIEF DESCRIPTION OF THE DRAWINGS

These and other objects, advantages and features of the invention will become apparent from the following description thereof taken in conjunction with the accompanying drawings in which:

FIG. 1 is a schematic view showing an example of the micro total analysis system;

FIG. 2 is a schematic view showing the first embodiment of the testing chip;

FIG. 3 is a schematic view for explaining the second example of the divided flow paths;

FIG. 4 is a schematic view for explaining the third example of the divided flow paths;

FIGS. 5(a) to 5(c) are schematic views showing the preferable shapes of the high flow path resistance portions and narrow flow paths;

FIG. 6 is a schematic view showing the second embodiment of the testing chip; and

FIGS. 7(a) to 7(c) are schematic views showing constitution examples of the micro-pump.

### DETAILED DESCRIPTION OF THE PREFERRED EMBODIMENTS

Hereinafter, the present invention will be explained on the basis of the embodiments drawn, though the present invention is not limited to the concerned embodiments. Further, in the drawings, to the same or similar parts, the same numerals are assigned and duplicated explanation will be omitted.

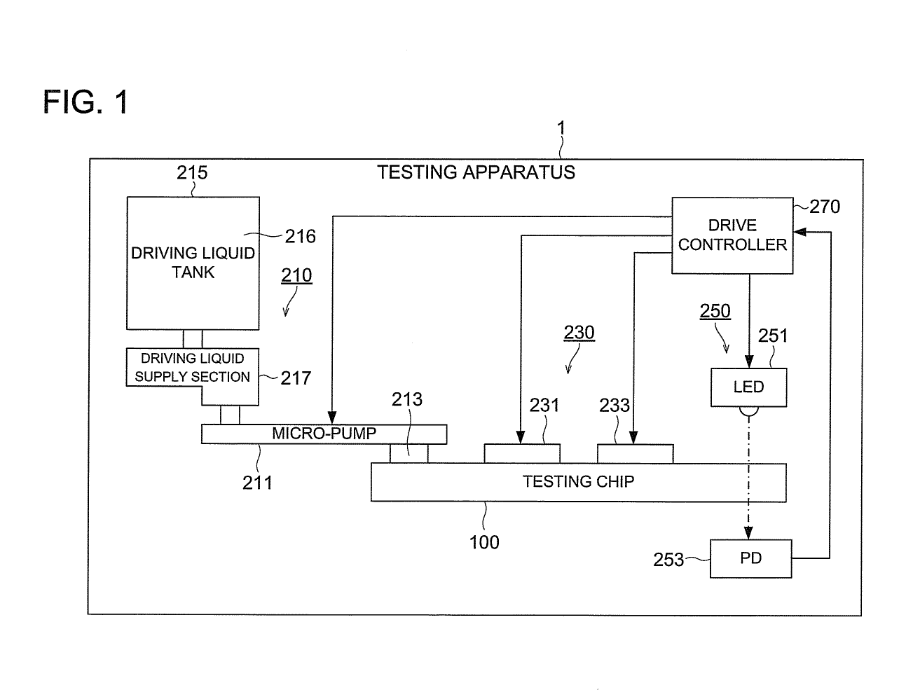

Firstly, the micro total analysis system of the present invention will be explained with reference to FIG. 1. FIG. 1 is a schematic view showing an example of the micro total analysis system.

In FIG. 1, a testing apparatus 1 which is a micro total analysis system of the present invention is composed of a testing chip 100 which is a micro total analysis chip of the present invention, a micro-pump unit 210 for feeding a liquid in the testing chip, a heating and cooling unit 230 for promoting and suppressing reaction in the testing chip, a detector 250 for detecting a target material included in a generated liquid obtained by reaction in the testing chip, and a drive controller 270 for driving, controlling, and detecting each unit in the testing apparatus. Here, the micro-pump unit 210 functions as a liquid feed apparatus of the present invention. As a liquid feed apparatus, in addition, an air pressure pump for feeding a liquid under an air pressure can be used.

The micro-pump unit 210 is composed of a micro-pump 211 for feeding a liquid, a chip connection section 213 for connecting the micro-pump 211 and testing chip 100, a driving liquid tank 215 for supplying a driving liquid 216 to be fed, and a driving liquid supply section 217 for supplying the driving liquid 216 from the driving liquid tank 215 to the micro-pump 211. The driving liquid tank 215 can be removed from the driving liquid supply section 217 to be exchanged to replenish the driving liquid 216. On the micro-pump 211, one or more pumps are formed and when a plurality of pumps are formed, they can be driven independently of each other or in link motion with each other.

The heating and cooling unit 230 is composed of a cooling section 231 composed of a Peltier element and a heating unit 233 composed of a heater. Needless to say, the heating element may be composed of a Peltier element. The detector 250 is composed of a light emitting diode (LED) 251 and a light receiving element (PD) 253 and detects optically a target material included in a generated liquid obtained by reaction in the testing chip.

The testing chip 100 is generally equivalent to a one referred to as an analysis chip or a micro reactor chip, wherein for example, a material of resin, glass, silicon, or ceramics is used, and therein, a fine flow path with a width and a height of several μm to several hundreds μm is formed by the microfine processing technique. The size of the testing chip 100 is generally several tens mm in length and width and several mm in height.

The testing chip 100 and micro-pump 211 are interconnected with the chip connection section 213 and when the micro-pump 211 is driven, various reagents and specimens stored in a plurality of storage sections in the testing chip 100 are fed by the driving liquid 216 flowing into the testing chip 100 from the micro-pump 211 via the chip connection section 213.

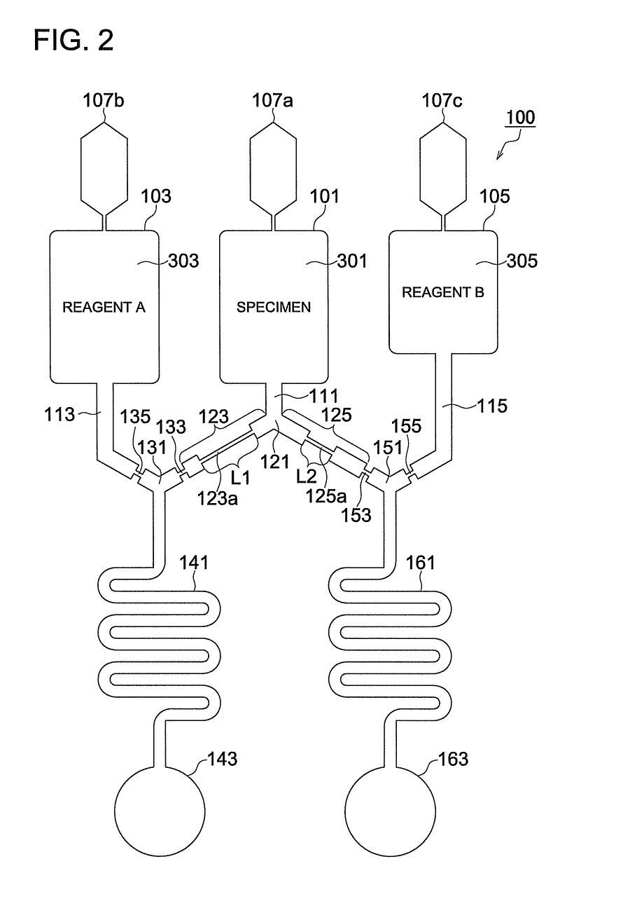

Next, the first embodiment of the testing chip 100 of the present invention will be explained by referring to FIG. 2. FIG. 2 is a schematic view showing the first embodiment of the testing chip 100. Here, a constitution example of the flow path for dividing a specimen into two flow paths, making the divided specimens react independently with two kinds of reagents, and executing a plurality of items of analysis and test will be explained.

In FIG. 2, in the testing chip 100, a specimen 301 is injected in a specimen storage section 101 and a reagent A 303 and a reagent B 305 are respectively injected in a reagent A storage section 103 and a reagent B storage section 105. On the upstream sides of the specimen storage section 101, reagent A storage section 103, and reagent B storage section 105, pump connection sections 107a, 107b, and 107c to which the micro-pump 211 is connected are installed and by the driving liquid 216 fed from the micro-pump 211, the specimen 301, reagent A 303, and reagent B 305 are fed downstream.

On the downstream side of the specimen storage section 101, a specimen main flow path 111 is installed and on the downstream side of the specimen main flow path 111, a branch portion 121 is installed. In this embodiment, the branch portion 121 is explained as branching into two ways, though the same may be said with multi-division such as division into 3 parts or more. On one downstream side of the branch portion 121, a first divided path 123 is installed, and on the first divided path 123, a first high flow path resistance portion with a length of L1 having a section of the flow path pressed narrower than the preceding and subsequent flow paths to increase the flow path resistance is installed, and similarly, on the other downstream side of the branch portion 121, a second divided path 125 is installed, and on the second divided path 125, a second high flow path resistance portion with a length of L2 is installed. Here, the branch portion 121, first divided path 123, and second divided path 125 function as a divided path of the present invention.

Further, the “flow path resistance” aforementioned is equivalent to the reciprocal of the flow rate of the liquid per unit pressure applied to the flow path, which can be obtained by measuring the flow rate when the liquid flows by applying a predetermined pressure at the entrance of the flow path and dividing the pressure by the flow rate. It will be described later in detail.

On the downstream side of the reagent A storage section 103, a reagent A main flow path 113 is installed, and the first divided path 123 and reagent A main flow path 113 are joined at a first joining portion 131 via water repellent valves 133 and 135, and on the downstream side of the first joining portion 131, a first mixing path 141 is installed, and on the downstream side of the first mixing path 141, a first detector 143 is installed. The specimen 301 and reagent A 303 joined at the first joining portion 131 are mixed in the first mixing path 141, are injected into the first detector 143, are reacted at the first detector 143, thus a reaction generated liquid is generated, and by the detector 250, a target material included in the reaction generated liquid is detected optically. The water repellent valves will be described later in detail.

Similarly, on the downstream side of the reagent B storage section 105, a reagent B main flow path 115 is installed, and the second divided path 125 and reagent B main flow path 115 are joined at a second joining portion 151 via water repellent valves 153 and 155, and on the downstream side of the second joining portion 151, a second mixing path 161 is installed, and on the downstream side of the second mixing path 161, a second detector 163 is installed. The specimen 301 and reagent B 305 joined at the second joining portion 153 are mixed in the second mixing path 161, are injected into the second detector 163, are reacted at the second detector 163, thus a reaction generated liquid is generated, and by the detector 250, a target material included in the reaction generated liquid is detected optically.

As an example, the mixing ratio of the reagent A 303 to the specimen 301 is assumed as 3:1, and the mixing ratio of the reagent B 305 to the specimen 301 is assumed as 1:1, and the volumes of the first detector 143 and second detector 163 are assumed as 4 nm³. In this case, the liquid feed amount of the reagent A 303 to the first detector 143 is 3 nm³ and the liquid feed amount of the specimen 301 to the first detector 143 is 1 nm³, and the liquid feed amount of the specimen 301 to the second detector 163 is 2 nm³, and the liquid feed amount of the reagent B 305 to the second detector 163 is 2 nm³.

To realize the aforementioned, it is necessary to divide and feed the specimen 301 at a flow rate ratio of 1:2. For that purpose, in the first divided path 123, a first high flow path resistance portion 123a with a length of L1 is installed, and in the second divided path 125, a second high flow path resistance portion 125a with a length of L2 is installed, and the ratio of the respective flow path resistances is set at 2:1. Concretely, the length L1 of the first high flow path resistance portion 123a is set at 5.0 mm and the length L2 of the second high flow path resistance portion 125a is set at 2.5 mm. The widths of the first high flow path resistance portion 123a and second high flow path resistance portion 125a are 50 μm and the depths thereof are 40 μm. Assuming the viscosity of the liquid as 1 mPa·s (equivalent to water at 20° C.), the flow path resistances of the first high flow path resistance portion 123a and second high flow path resistance portion 125a are respectively 40×10¹² (N·s/m⁵) and 20×10¹² (N·s/m⁵).

Here, the “flow path resistance” will be described in detail. The “flow path resistance” is equivalent to the reciprocal of the flow rate of the liquid per unit pressure applied to the flow path, which can be obtained by measuring the flow rate when the liquid flows by applying a predetermined pressure at the entrance of the flow path and dividing the pressure by the flow rate. Particularly, as in the example aforementioned, when the flow path is narrow and long and in the flow of the liquid in the flow path, the laminar flow is dominant, the flow path resistance R can be calculated by the following formula.

$$\begin{matrix}Formula 1 & \\ R = \int \frac{32 \times η}{S \times {φ}^{2}} d L & ( Formula 1 )\end{matrix}$$

Where, η indicates viscosity of the liquid, S a sectional area of the flow path, Φ an equivalent diameter of the flow path, and L a length of the flow path. Further, the equivalent diameter Φ, in the section of a rectangle with a width of a and a height of b, is indicated as shown below.

Φ=(a×b)/{(a+b)/2} (Formula 2)

Next, the liquid feeding procedure in the aforementioned first embodiment of the testing chip 100 will be explained. Firstly, the three micro-pumps 211 connected to the pump connection sections 107a, 107b, and 107c are driven simultaneously under a comparatively weak pressure such as about 2 kPa. At this time, the specimen 301, reagent A 303, and reagent B 305 are fed respectively downstream and when they reach the water repellent valves 133, 135, 153, and 155, the liquid feed is stopped by the liquid holding force due to the water repellency of the water repellent valves. Further, the width of the water repellent valves in this example is 25 μm and the liquid holding force of the water repellent valves is about 4 kPa.

Here, the water repellent valve is a narrow flow path which is hydrophobic and is narrow in width and when a liquid is fed under lower than a predetermined pressure, by the water repellent force of the narrow flow path, the flow of the liquid can be stopped in place. In the example aforementioned, at time of liquid feed by the micro-pumps 211, via the water repellent valves 133 and 135 and the water repellent valves 153 and 155, the specimen 301 and reagent A 303 and the specimen 301 and reagent B 305 can be led to the first joining portion 131 and the second joining portion 151, thus the liquid feed timings can coincide with each other, and the specimen 301 and reagent A 303 and the specimen 301 and reagent B 305 can be mixed at an accurate mixing ratio.

Next, when pressure over the liquid holding force of the water repellent valves (for example, 10 kPa or higher) is applied simultaneously from the three micro-pumps 211, the specimen 301 and reagent A 303 and the specimen 301 and reagent B 305 are simultaneously joined respectively at the first joining portion 131 and the second joining portion 151, flow into the first mixing path 141 and the second mixing path 161, and are injected into the first detector 143 and the second detector 163.

At this time, the specimen 301, at the branch portion 121, according to the reciprocal of the ratio of the resistance between the first high flow path resistance portion 123a and the second high flow path resistance portion 125a, is divided into two parts at a division ratio of 1:2 and is fed. The reagent A 303 and reagent B 305 can be fed at an optional liquid feed amount under the liquid feed pressure of the micro-pumps 211, so that they can be mixed at the desired liquid feed amount as aforementioned and at a desired mixing ratio.

Further, when the testing chip has a sectional size of the flow path on the order of several tens μm, the interaction force such as the capillary force acting between the inner wall surface of the flow path and the liquid greatly influences liquid feed. Such interaction force is extremely easily influenced by the surface conditions of the flow path such as roughness of the inner wall surface of the flow path and foreign substances on the inner wall surface. Therefore, even if it is intended to divide the liquid at the desired division ratio at the branch portion, due to the effect of this interaction force, the division ratio is varied easily.

Particularly, as in this embodiment, on both downstream sides of the first divided path 123 and second divided path 125 which are divided into two parts, the water repellent valves 133 and 153 are installed, and when the two stop the liquid once at the water repellent valves 133 and 153 and then simultaneously at the desired timing, refeed the liquid at the predetermined division ratio, due to characteristic variations of the water repellent valves 133 and 153, only the water repellent valve of either of the divided paths may permit the liquid to pass earlier.

For example, when the specimen 301 passes the water repellent valve 133, if the end (hereinafter, referred to as the meniscus portion) of the specimen 301 is kept held under the liquid holding pressure by the water repellent force of the water repellent valve 153, thereafter, the specimen 301 flows only in the first divided path 123 of the water repellent valve 133, thus a phenomenon that the specimen 301 of the second divided path 125 of the water repellent valve 153 cannot pass indefinitely the water repellent valve 153 may occur.

As a countermeasure for preventing the phenomenon aforementioned, according to the present invention, assuming any flow path resistance of the first high flow path resistance portion 123a and second high flow path resistance portion 125a, for example, the flow path resistance of the first high flow path resistance portion 123a as R, the flow rate of the specimen 301 of the first divided flow path 123 including the first high flow path resistance portion 123a as Q, and the upper limit of the liquid holding pressure of the water repellent valve 153 existing in the second divided flow path 125 as P, it has been found that the phenomenon can be solved by the following setting

R×Q>P (Formula 3)

The same may be said with the second high flow path resistance portion 125a.

Here, R×Q is equivalent to the pressure difference between the end of the first high flow path resistance portion 123 on the upstream side and the end thereof on the downstream side. The pressure of the meniscus portion of a liquid on the downstream side during flowing is almost equal to the air pressure, so that it means that the pressure difference between the end of the first high flow path resistance portion 123a on the upstream side and the air pressure is almost R×Q. In such a case, as viewed from the connection of the flow paths, when the water repellent valve 153 holds the meniscus portion of the specimen 301, the pressure difference R×Q is applied to both ends of the water repellent valve 153. Therefore, if the value of R×Q of the first high flow path resistance portion 123a is the liquid holding pressure P of the water repellent valve 153 or higher, the problem aforementioned is solved, and the liquid flows out immediately from the water repellent valve 153 and is fed at the desired division ratio.

As a concrete example, the flow path resistance R of the first high flow path resistance portion 123a is 40×10¹² (N·s/m⁵) and the flow rate Q flowing through the flow path including the first high flow path resistance portion 123a is 0.15×10⁻⁹ (m³/s). At this time, R×Q=6 kPa is held and it is set at a value larger than the upper limit P (=4 kPa) of the liquid holding pressure of the water repellent valve 153.

Further, when the liquid feed pressure of the micro-pumps 211 starts up slow, a lot of time is required for the flow rate Q to reach a predetermined value, so that during the period, the value of R×Q becomes smaller than a supposed value, thus a problem arises that the liquid is fed only in one flow path before reaching the predetermined value. Therefore, it is preferable to make the start-up time of liquid feed of the micro-pumps 211 as short as possible.

According to the aforementioned first embodiment of the testing chip 100 of the present invention, the ratio of the flow path resistance of each of the divided flow paths branched into a plurality of parts is set at almost the same as the reciprocal of the predetermined division ratio of the liquid which is divided and fed in each of the divided flow paths, thus divided flow paths branched into a plurality of parts for accurately dividing and feeding a liquid such as a specimen or a reagent at a predetermined division ratio can be realized and the time required for analysis can be shortened by producing a plurality of reactions in parallel.

Furthermore, according to the aforementioned first embodiment of the testing chip 100 of the present invention, when the water repellent valves are installed in the divided paths and the flow path resistance R of the high flow path resistance portion is set so as to satisfy Formula 3, the phenomenon that only the water repellent valve in one divided path permits a liquid to pass earlier and no liquid passes indefinitely the other divided path can be prevented, so that divided flow paths branched into a plurality of parts for accurately dividing and feeding a liquid such as a specimen or a reagent at a predetermined division ratio can be realized and the time required for analysis can be shortened by producing a plurality of reactions in parallel.

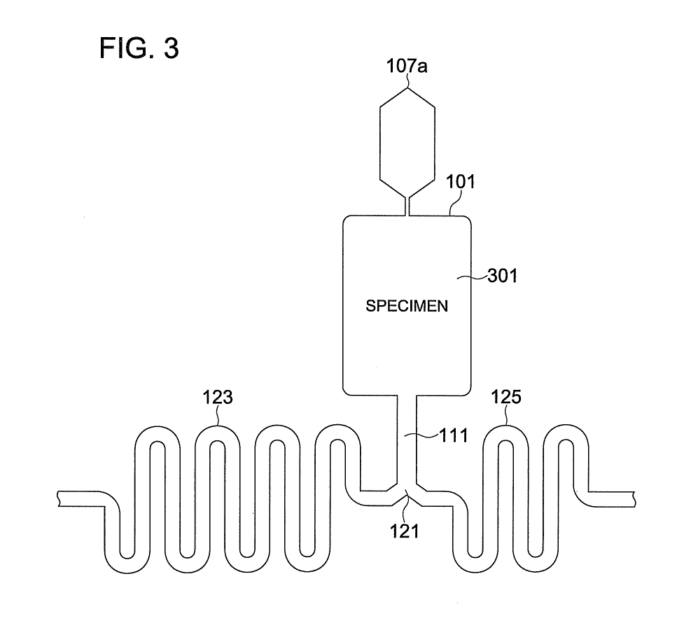

Next, the second example of the divided paths of the first embodiment of the testing chip 100 will be explained by referring to FIG. 3. FIG. 3 is a schematic view for explaining the second example of the divided flow paths. In FIG. 3, the parts equivalent to the pump connection section 107a, specimen storage section 101, specimen main flow path 111, branch portion 121, first divided path 123, first high flow path resistance portion 123a, second divided path 125, and second high flow path resistance portion 125a which are shown in FIG. 2 are shown.

If the flow path resistances of the first divided path 123 and second divided path 125 can be set to the predetermined values aforementioned, there is no need to provide the flow path whose width is narrowed deliberately as a “high flow path resistance portion”. Therefore, in the example shown in FIG. 3, on the downstream side of the branch portion 121, the first high flow path resistance portion 123a and second high flow path resistance portion 125a are not installed, and the first divided path 123 and second divided path 125 having the same width as that of the other flow paths and a longer length than that of the other flow paths are installed, and the flow path resistances are adjusted depending on the length.

In this example, the length of the first divided path 123 is about two times of the length of the second divided path 125, thus the flow path resistance of the first divided path 123 can be almost double the flow path resistance of the second divided path 125.

In the aforementioned second example of the divided paths, the divided paths are made longer, and the flow path resistances are set at the predetermined values, thus without using the high flow path resistance portions, the same function as that of the example using the first high flow path resistance portion 123a and second high flow path resistance portion 125a which are shown in FIG. 2, can be performed and the similar effect can be obtained.

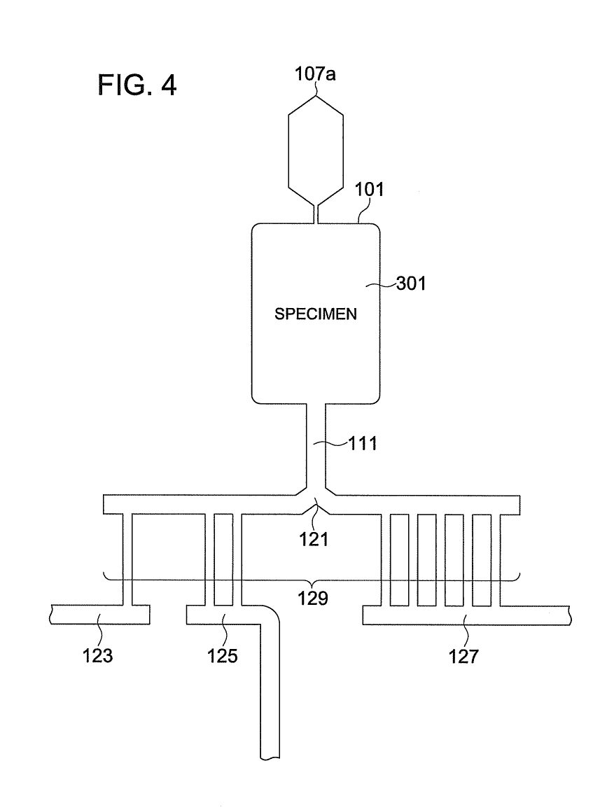

Next, the third example of the divided paths of the first embodiment of the testing chip 100 will be explained by referring to FIG. 4. FIG. 4 is a schematic view for explaining the third example of the divided flow paths. In FIG. 4, an example of the divided paths for dividing the specimen 301 into three parts of 1:2:5 is shown and the drawn range, similarly to FIG. 3, includes the parts equivalent to the pump connection section 107a, specimen storage section 101, specimen main flow path 111, branch portion 121, first divided path 123, first high flow path resistance portion 123a, second divided path 125, and second high flow path resistance portion 125a which are shown in FIG. 2.

In FIG. 4, on the downstream side of the specimen storage section 101, the specimen main flow path 111 is installed and on the downstream side of the specimen main flow path 111, the branch portion 121 is installed. On the downstream side of the branch portion 121, eight narrow flow paths 129 which are in the same width and length are installed and on the downstream side of the eight narrow flow paths 129, the first divided path 123 connected to one narrow flow path 129, the second divided path 125 connected to two narrow flow paths 129, and third divided path 127 connected to five narrow flow paths 129 are installed. In this case, the division ratio of the specimen 301 which are divided into the first divided path 123, second divided path 125, and third divided path 127 is 1:2:5.

In the aforementioned third example of the divided paths, a plurality of narrow flow paths 129 in the same shape are installed in parallel, and the narrow flow paths 129 are connected to form divided paths in parallel according to a necessary division ratio, thus a very highly precise division ratio can be realized, and highly precise analysis and test equivalent to or higher than the examples shown in FIGS. 2 and 3 can be realized.

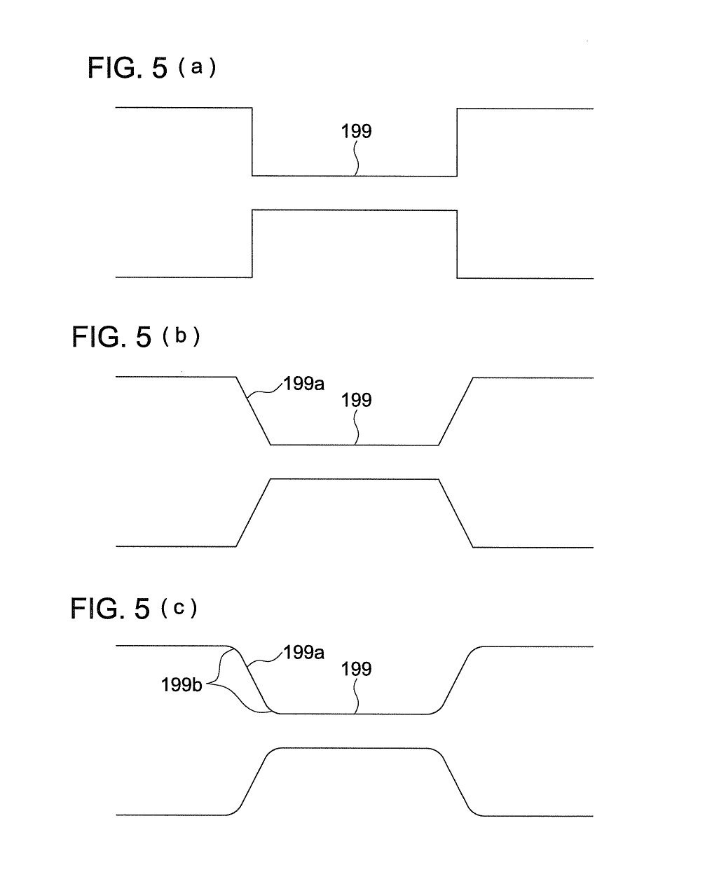

Next, the preferable shapes of the high flow path resistance portions shown in FIG. 2 and the narrow flow paths shown in FIG. 4 will be explained by referring to FIG. 5. FIG. 5 are schematic views showing the preferable shapes of the high flow path resistance portions and narrow flow paths.

The high flow path resistance portions shown in FIG. 2 and narrow flow paths 199 of the narrow flow paths shown in FIG. 4 may be the same in depth as the preceding and subsequent flow paths or only the portions may be changed in depth. When the depth is reduced, the flow path resistance is increased in correspondence to it.

Further, the outlet and inlet of the high flow path resistance portion and narrow flow path, as shown in FIG. 5(a), may be shaped so as to have a level difference in the flow path width, though in the shape shown in FIG. 5(a), in relation to the wettability between the liquid and the flow path surface at the outlet and inlet, the meniscus portion of the liquid is apt to be held easily, so that there are possibilities that the function similar to the “water repellent valve” aforementioned may be performed. Therefore, for the outlet and inlet of the high flow path resistance portion and narrow flow path, particularly the outlet, a shape that as shown in FIG. 5(b), no sudden level difference is formed, and an inclined portion 199a is provided, and the width thereof is changed slowly or a shape that as shown in FIG. 5(c), in addition to the inclined portion 199a, a curved surface 199b that the corners of the inclined portion 199a are rounded off smoothly is provided is preferable.

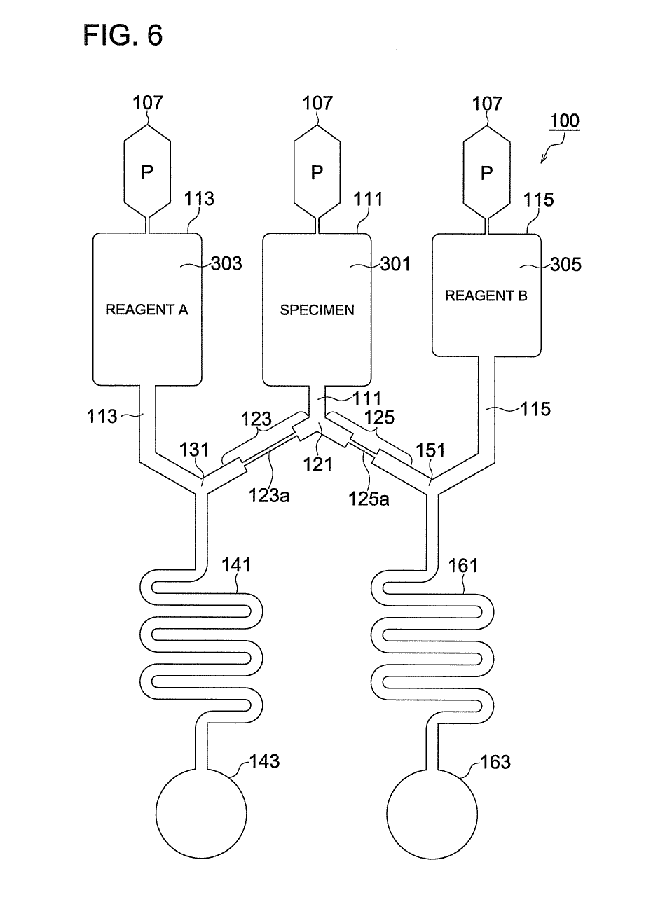

Next, the second embodiment of the testing chip 100 of the present invention will be explained by referring to FIG. 6. FIG. 2 is a schematic view showing the second embodiment of the testing chip 100. Here, a constitution example that in the first embodiment shown in FIG. 2, the water repellent valves 133, 135, 153, and 155 are not installed will be explained.

In FIG. 2, when the water repellent valves 133, 135, 153, and 155 are installed, it is explained that the flow path resistance R of the high flow path resistance portions, the flow rate Q of the divided flow paths including the high flow path resistance portions, and the upper limit P of the liquid holding pressure of the water repellent valves existing in the other divided flow paths must satisfy the relation of Formula 3, though as shown in FIG. 6, even if the water repellent valves 133, 135, 153, and 155 are installed, the flow path resistance R is desirably the predetermined value or larger.

The reason is that when the testing chip has a sectional size of the flow path on the order of several tens μm, regardless of existence of the water repellent valve, under the influence of the interaction force such as the capillary force acting between the inner wall surface of the flow path and the liquid, it is kept unchanged that the division ratio of the liquid is varied easily. The capillary force Pc of the flow path, assuming the sectional area of the flow path as S, the peripheral length of the section as L, the surface tension of a liquid to be fed as σ, and the contact angle between the inner wall surface of the flow path and the meniscus portion of the liquid to be fed as θ, can be expressed by the formula indicated below.

Pc=(σ·L/S)×cos θ (Formula 4)

At this time, cos θ is very variable due to the roughness of the inner wall surface of the flow path and foreign substances on the inner wall surface. To execute division liquid feed with precision producing no effect on analysis and test regardless of the variations, it is desirable that a pressure difference Pd applied to both ends of the high flow path resistance portion is at least the maximum value σ·L/S of the capillary force Pc aforementioned or larger. The pressure difference Pd applied to both ends of the high flow path resistance portion, as mentioned above, is almost equal to the product R×Q of the flow path resistance R of the other high flow path resistance portion and the flow rate Q, so that it is desirable to set the flow path resistance R of each high flow path resistance portion so as to satisfy the relation of:

R×Q>σ·L/S (Formula 5)

In the concrete example shown in FIG. 2, the flow path resistance R of the first high flow path resistance portion 123a is 40×10¹² (N·s/m⁵) and the flow rate Q flowing through the first divided path 123 including the first high flow path resistance portion 123a is 0.15×10⁻⁹ (m³/s). At this time, the value of R×Q is 6 kPa. In the second divided path 125, the flow path section has a width of 200 μm and a depth of 250 μm, and the surface tension a of a liquid to be fed is almost the same as that of water such as 73 (mN/m), so that the maximum value σ·L/S of the capillary force Pc becomes about 1.3 kPa, thus the relation of Formula 5 is held.

According to the aforementioned second embodiment of the testing chip 100 of the present invention, when the water repellent valves are not installed in the divided paths, the flow path resistance R of the high flow path resistance portions is set so as to satisfy Formula 5, thus without influenced by variations of the division ratio of the liquid caused by the interaction force such as the capillary force acting between the inner wall surface of the flow path and the liquid, division liquid feed can be executed at a stable division ratio and by producing a plurality of reactions in parallel, the time required for analysis can be shortened.

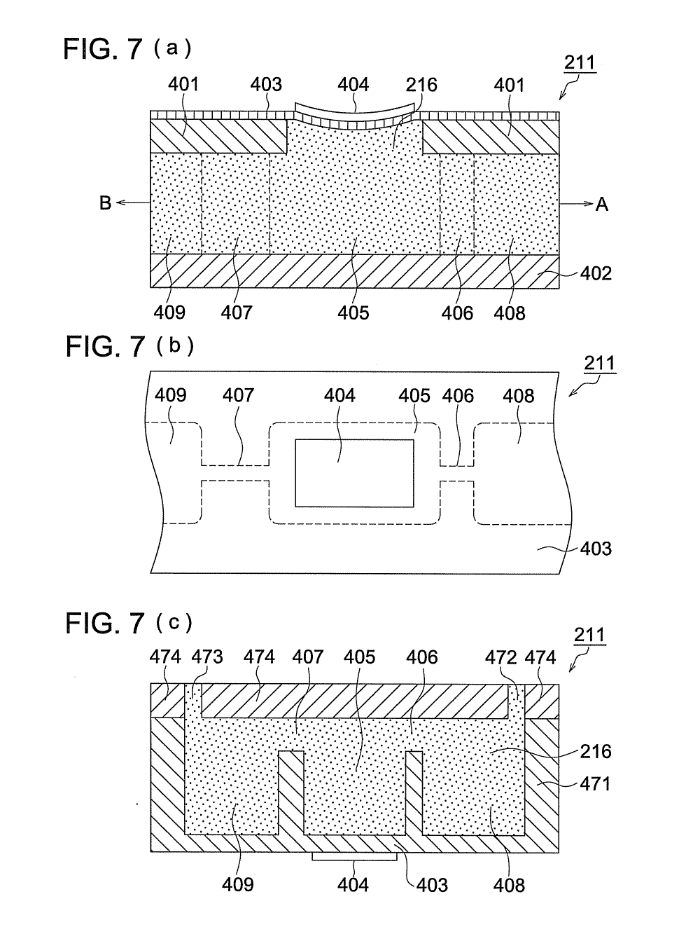

Next, an example of the micro-pumps 211 used for liquid feed in the aforementioned first and second embodiments of the testing chip 100 will be explained by referring to FIG. 7. For the micro-pumps 211, various pumps such as a check valve type pump having a check valve installed at the outlet and inlet hole of the valve chamber provided with an actuator can be used, though a piezo-electric pump is used preferably. FIG. 7 are schematic views showing constitution examples of the micro-pump 211, and FIG. 7(a) is a cross sectional view showing an example of the piezo-electric pump, and FIG. 7(b) is a top view thereof, and FIG. 7(c) is a cross sectional view showing another example of the piezo-electric pump.

In FIGS. 7(a) and 7(b), the micro-pump 211 is equipped with a substrate 402 including a first liquid chamber 408, a first flow path 406, a pressurizing chamber 405, a second flow path 407, and a second liquid chamber 409, an upper substrate 401 laminated on the substrate 402, a diaphragm 403 laminated on the upper substrate 401, a piezo-electric element 404 laminated on the side of the diaphragm 403 opposite to the pressurizing chamber 405, and a driving section not drawn for driving the piezo-electric element 404. The driving section and the two electrodes on both surfaces of the piezo-electric element 404 are connected by wires such as a flexible cable and through the wires, by the drive circuit of the driving section, the drive voltage is impressed to the piezo-electric element 404. At time of driving, the first liquid chamber 408, first flow path 406, pressurizing chamber 405, second flow path 407, and second liquid chamber 409 are internally filled with the driving liquid 216.

As an example, as a substrate 402, a photosensitive glass substrate with a thickness of 500 μm is used and etched up to a depth of 100 μm, thus the first liquid chamber 408, first flow path 406, pressurizing chamber 405, second flow path 407, and second liquid chamber 409 are formed. The first flow path 406 has a width of 25 μm and a length of 20 μm. Further, the second flow path 407 has a width of 25 μm and a length of 150 μm.

The upper substrate 401 which is a glass substrate is laminated on the substrate 402, thus the tops of the first liquid chamber 408, first flow path 406, second liquid chamber 409, and second flow path 407 are formed. The part of the upper substrate 401 touching the top of the pressurizing chamber 405 is processed and pierced by etching.

On the top of the upper substrate 401, the diaphragm 403 composed of a thin glass plate with a thickness of 50 μm is laminated and thereon, the piezo-electric element 404 composed of, for example, titanate zirconate (PZT) ceramics with a thickness of 50 μm is pasted. By the drive voltage from the driving section, the piezo-electric element 404 and diaphragm 403 pasted thereon vibrate, thus the volume of the pressurizing chamber 405 is increased or decreased.

The first flow path 406 and second flow path 407 are the same in width and depth, and the second flow path 407 is longer than the first flow path 406, and in the first flow path 406, when the pressure difference is increased, a turbulent flow is generated at the outlet and inlet of the flow path and around it, thus the flow resistance is increased. On the other hand, in the second flow path 407, the flow path is longer, so that even if the pressure difference is increased, a laminar flow is apt to be generated and compared with the first flow path 406, the change rate of the flow path resistance to the change in the pressure difference is reduced. Namely, depending on the magnitude of the pressure difference, the relationship of easiness of flow of the liquid between the first flow path 406 and the second flow path 407 is changed. By use of it, the drive voltage waveform to the piezo-electric element 404 is controlled, thus the liquid is fed.

For example, by the drive voltage to the piezo-electric element 404, the diaphragm 403 is shifted quickly inward the pressurizing chamber 405, and the volume of the pressurizing chamber 405 is reduced by applying a high pressure difference, and then the diaphragm 403 is shifted slowly outward from the pressurizing chamber 405, and the volume of the pressurizing chamber 405 is increased by applying a low pressure difference, thus the liquid is fed from the pressurizing chamber 405 toward the second liquid chamber 409 (the direction B shown in FIG. 7(a)).

Inversely, the diaphragm 403 is shifted quickly outward from the pressurizing chamber 405, and the volume of the pressurizing chamber 405 is increased by applying a high pressure difference, and then the diaphragm 403 is shifted slowly inward from the pressurizing chamber 405, and the volume of the pressurizing chamber 405 is reduced by applying a low pressure difference, thus the liquid is fed from the pressurizing chamber 405 toward the first liquid chamber 408 (the direction A shown in FIG. 7(a)).

Further, the difference of the change rate of the flow path resistance to the change in the pressure difference between the first flow path 406 and the second flow path 407 may not be always necessarily varied with the difference in the flow path length and may be based on another shape difference.

In the micro-pump 211 structured as mentioned above, by changing the drive voltage and frequency for the pump, the desired liquid feed direction and liquid feed speed can be controlled. Although not drawn in FIGS. 7(a) and 7(b), in the first liquid chamber 408, a port connected to the driving liquid tank 215 is installed, and the first liquid chamber 408 plays a roll of “reservoir” and is supplied with the driving liquid 216 by the port from the driving liquid tank 215. The second liquid chamber 409 forms the flow path of the micro-pump unit 210, and the chip connection section 213 is installed ahead it and is connected to the testing chip.

In FIG. 7(c), the micro-pump 211 is composed of a silicone substrate 471, the piezo-electric element 404, a substrate 474, and flexible wires not drawn. The silicone substrate 471 is obtained by processing a silicone wafer in a predetermined shape by the photolithographic technique and by etching, the pressurizing chamber 405, diaphragm 403, first flow path 406, first liquid chamber 408, second flow path 407, and second liquid chamber 409 are formed. At time of driving, the pressurizing chamber 405, first flow path 406, second flow path 407, first liquid chamber 408, and second liquid chamber 409 are internally filled with the driving liquid 216.

On the substrate 474, a port 472 is installed on the upper part of the first liquid chamber 408, and a port 473 is installed on the upper part of the second liquid chamber 409, and for example, when the micro-pump 211 is installed separately from the testing chip 100, it can be interconnected with the pump connection section of the testing chip 100 via the port 473. For example, the ports 472 and 473 stack vertically the substrate 474 perforated and the neighborhood of the pump connection section of the testing chip 100, thus the micro-pump 211 can be connected to the testing chip 100.

Further, as mentioned above, the micro-pump 211 is obtained by processing a silicone wafer in a predetermined shape by the photolithographic technique, so that on one silicone substrate, a plurality of micro-pumps 211 can be formed. In this case, to the port 472 on the opposite side of the port 473 connected to the testing chip 100, the driving liquid tank 215 is desirably connected. When there are a plurality of micro-pumps 211 installed, the ports 472 therefor may be connected to the common driving liquid tank 215.

The micro-pump 211 aforementioned is small-sized, is given a small dead volume due to the wire from the micro-pump 211 to the testing chip 100, is changed little due to pressure, and can be applied instantaneously with precise discharge pressure control, so that precise liquid feed control can be executed by the drive controller 270.

As mentioned above, according to the present invention, the ratio of the flow path resistance of each of the divided flow paths branched into a plurality of parts is set at almost the same as the reciprocal of the predetermined division ratio of the liquid which is divided and fed in each of the divided flow paths, thus a micro total analysis chip and a micro total analysis system capable of realizing divided flow paths branched into a plurality of parts for accurately dividing and feeding a liquid such as a specimen or a reagent at a predetermined division ratio and shortening the time required for analysis by producing a plurality of reactions in parallel can be provided.

Furthermore, according to the aforementioned first embodiment of the testing chip 100 of the present invention, when the water repellent valves are installed in the divided paths and the flow path resistance R of the high flow path resistance portion is set so as to satisfy Formula 3, the phenomenon that only the water repellent valve in one divided path permits a liquid to pass earlier and no liquid passes indefinitely the other divided path can be prevented, so that a micro total analysis chip and a micro total analysis system capable of realizing divided flow paths branched into a plurality of parts for accurately dividing and feeding a liquid such as a specimen or a reagent at a predetermined division ratio and shortening the time required for analysis by producing a plurality of reactions in parallel can be provided.

According to the present invention, by setting the flow path resistance of the high flow path resistance portion so that the relationship between the flow path resistance of the high flow path resistance portion of the divided flow paths and the flow rate of the divided flow paths including the concerned high flow path resistance portion, sectional area of the other divided flow paths, peripheral length of the section, and surface tension of the liquid to be fed satisfies a predetermined relationship, a micro total analysis chip and a micro total analysis system capable of realizing divided flow paths branched into a plurality of parts for accurately dividing and feeding a liquid such as a specimen or a reagent at a predetermined division ratio and shortening the time required for analysis by producing a plurality of reactions in parallel can be provided.

The detailed constitution and detailed operation of each component composing the micro total analysis chip and micro total analysis system relating to the present invention can be modified properly within a range which is not deviated from the nature of the invention.

What is claimed is:

1. A micro total analysis chip comprising:
a main flow path for feeding a liquid; and
a divided flow path branched into a plurality of divided paths for dividing and feeding the liquid fed from the main flow path at a predetermined division ratio, each divided path of the plurality of divided paths having a high flow path resistance portion comprising a narrowed down flow path narrower than a preceding part and a subsequent part of the each divided path,
wherein a flow path resistance R of the high flow path resistance portion of a first divided path in any of the plurality of divided paths satisfies an expression of:
R×Q>σ×L/S
where, Q is a flow rate of the first divided path,
S is a sectional area and L is a sectional circumferential length of a flow path of other divided path of the plurality of divided paths than the first divided path, and
σ is a surface tension of the liquid.

2. The micro total analysis chip of claim 1, wherein each of the plurality of divided paths has at least one water repellent valve,
wherein the flow path resistance R of the high flow path resistance portion of the first divided path in any of the plurality of divided paths satisfies an expression of:
R×Q>P
where, Q is the flow rate of the first divided path,
P is an upper limit of a liquid holding pressure of the water repellent valve in other divided path of the plurality of divided paths than the first divided path.

3. A micro total analysis system comprising:
a micro total analysis chip of claim 1;
a liquid feed apparatus connected to the micro total analysis chip for feeding a liquid in the micro total analysis chip; and
a detector for detecting a target material generated on the micro total analysis chip.

4. A micro total analysis system comprising:
a micro total analysis chip of claim 2;
a liquid feed apparatus connected to the micro total analysis chip for feeding a liquid in the micro total analysis chip; and
a detector for detecting a target material generated on the micro total analysis chip.
